(19) United States
(12) Patent Application Publication
(10) Pub. No.: US 2003/0152607 A1
(43) Pub. Date: Aug. 14, 2003

(54) Caloric management system and method with voice recognition

(76) Inventor: James R. Mault, Evergreen, CO (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/256,667

(22) Filed: Sep. 27, 2002

Related U.S. Application Data
(60) Provisional application No. 60/356,533, filed on Feb. 13, 2002.

(51) Int. Cl. G06F 17/60; G10L 15/00; G10L 17/00; A61K 47/00

(52) U.S. Cl. 424/439; 704/246; 705/2

(57) ABSTRACT

A caloric management system and method for assisting a plurality of users to implement a caloric management program personal to each user by providing a personal user device for each user, and a data processor at a remote location in communication with the personal user devices. Each personal user device includes a microphone, a personal identification device, a display, and a two-way communication system with respect to the data processor at the remote location. The data processor at the remote location includes a food lookup table containing data representing various quantified food items and the caloric intake value of each. A voice recognition circuit, preferably in the user data file for each personal user device in the data processor at the remote location, analyzes speech messages by the users to identify consumed food items. Each user data file at the remote location also includes a computer for computing the total caloric intake value of the consumed food items and other information for transmission to the respective personal user device for display thereon.

### RELATED APPLICATIONS

The present application is related to, and claims the priority date of, Provisional Application Serial No. 60/356,533 filed Feb. 13, 2002, and incorporates by reference the entire contents of such application. The present application is also related to co-pending application Ser. No. 09/976,967 filed Oct. 12, 2001, assigned to the same assignee as the present application, and incorporates herein by reference the entire contents of that application.

### FIELD OF THE INVENTION

The present application relates to a system and method for assisting users to implement a caloric management program personal to each user.

### BACKGROUND OF THE INVENTION

Good health and weight control are of considerable interest to a large number of people. Many people engage in conventional weight control programs usually based on a restricted calorie diet in order to achieve weight loss. Physical activity may also be included in a calorie management system of a weight control program.

A caloric management system allows a person to compare caloric expenditure with caloric intake in the form of food and beverages consumed by the person. Caloric expenditure has two components: A larger component is related to the energy expended in resting metabolic processes; and a smaller component is related to the energy expended in physical activity. Total energy expenditure (TEE) is the sum of resting energy expenditure (REE), a product of resting metabolic rate (RMR) and the time period of interest, and activity-related energy expenditure (AEE); that is:

TEE=REE+AEE

Calorie balance is defined in terms of the difference between TEE and the caloric intake of the person.

A variety of systems exist for recording a user's food and beverage intake in order to determine caloric or other nutritional values consumed for use in weight control and the like. Williamson U.S. Pat. Nos. 4,891,756 and 5,704,350 disclose systems using a computer or a PDA for inputting the identification and quantities of consumed foods and beverages. However, the task of inputting the identification and quantities of consumed foods and beverages using a computer or a PDA of the type shown in Williamson U.S. Pat. Nos. 4,891,756 and 5,704,350 involves a fair degree of discipline, and many persons who would desire a record of their caloric or nutritional input quickly abandon the recording process after starting it. The simpler the recording task can be made, the larger will be the percentage of users who will adhere to it.

European published Patent Application EP 1,122,524 describes a similar portable user device having voice recognition circuitry for enabling the food items to be inputted into the device. However, each such user device includes not only the voice recognition circuitry, but also the data processing circuitry for processing this information. Such a construction substantially increases the cost of each personal user device, and thereby makes such devices less affordable to a large segment of the population. The simpler the individual user device, the larger will be the segment of the population that will be able to afford it, and thereby to use the caloric management system.

### OBJECTS AND BRIEF SUMMARY OF THE INVENTION

An object of the present invention is to provide a caloric management system and method having advantages in the above respects.

According to one aspect of the present invention, there is provided a caloric management system for assisting a plurality of users to implement a caloric management program personal to each user, comprising: (a) a plurality of personal user devices each including: (i) input means including a microphone for converting speech sounds by the respective user to electrical signals for outputting from the respective personal user device; (ii) a personal identification device for applying a personal identification signal in the output of the respective personal user device; (iii) a display for displaying information; and (iv) a two-way communication system for transmitting the output of the personal user device to a remote location serving the plurality of personal user devices, and for receiving information from the remote location; (b) a voice recognition circuit for analyzing the outputs of the microphones of the plurality of personal user devices to identify quantified food items represented thereby; and (c) a data processor at the remote location including: (i) a two-way communication system for receiving the output from each personal user device; (ii) a food lookup table containing data representing a number of quantified food items and the caloric intake value of each such food item; and (iii) a user data file for each personal user device, each of the user data files including a computer for computing the total caloric intake value of the quantified food items identified in a particular time period from the respective personal user device and for transmitting information regarding the total caloric intake value to the respective personal user device for display thereon.

In the preferred embodiment of the invention described below, the voice recognition circuit is included in the data processor at the remote location. More particularly, the voice recognition circuit is user-dependent, and is included in each of the user data files in the data processor at the remote location. A less advantageous alternative would be to include a voice recognition circuit in each of the personal user devices, but this would substantially increase the cost of such user devices, and thereby make the caloric management system less available.

According, to further features included in the described preferred embodiment, the input means in each of the personal user devices also enables the respective user to input a caloric management program into its respective user data file in the data processor at the remote location. The information transmitted by the computer in the respective user data file also includes information comparing the total caloric intake value for the respective time period with the caloric management program inputted into the respective user data file.

According to still further features in the described preferred embodiment, at least some of the user data files in the data processor at the remote location also include an activity lookup table containing data representing a number of physical activities and the caloric expenditure value of each such physical activity when performed by the respective user per unit time. The voice recognition circuit in each user data file having an activity lookup table also analyzes the output from the respective personal user device to identify physical activities and the period of time the user was involved therein. The computer in each user data file having an activity lookup table also computes the caloric expenditure value for the particular physical activity and time period, and includes the caloric expenditure value in the information transmitted to the respective personal user device.

In addition, in the described preferred embodiment the input means of each personal user device also enables inputting a resting metabolism rate which is converted to electrical metabolism signals also transmitted to the data processor at the remote location. The computer in each user data file having an activity lookup table also analyzes the electrical metabolism signals for computing the caloric expenditure value corresponding to the resting metabolism rate for the respective time period, and includes such information in computing the caloric expenditure value transmitted to the respective personal user device. In one described embodiment, such input means includes a connector for an indirect calorimeter for inputting the resting metabolism rate. In another described embodiment, the input means enables inputting information regarding the sex, age and weight of the user to enable the computer within the data processor at the remote location to compute an estimate of the resting metabolism rate from the inputted information, e.g., according to the known Harris-Benedict Equation.

According to still further features in a described preferred embodiment, the input means in each of the personal user devices also enables inputting a caloric management program into its respective user data file in the data processor at the remote location. In such case, the computer in the respective user data file at the remote location also generates diet and/or activity recommendations which are included in the information transmitted to the respective personal user device to better enable the user to implement the inputted caloric management program.

According to still further features in a described preferred embodiment, the data processor at the remote location also includes an audio prompt generator which, upon receiving a transmission from a personal user device, enables the data processor to transmit back to the respective personal user device an inquiry regarding the transmission by the personal user device, which inquiry requires a response from the respective personal user device.

In a described preferred embodiment, at least some of the personal user devices are coupleable to portable hand-carried recording devices which include audio recorders for recording the identification of the quantified food items as consumed by the user and for downloading into the respective personal user devices at convenient time intervals. The two-way communication system between the personal user devices and the data processor at the remote location is preferably via the Internet.

According to another aspect of the present invention, there is provided a method of assisting a plurality of users to implement a caloric management program personal to each user by using the above-described caloric management system.

The present invention thus relieves the user of such a caloric management system of the manual task of entering information by means of a keyboard, stylus or the like, and allows voice messages concerning the foods and beverages consumed, and also exercise activity performed, to be used for recording this information. Since most of the data processing functions, as well as the data storage functions, are performed in the data processor at the remote location for all the personal user devices, each personal user device may therefore be constructed very simply and inexpensively, thereby enabling a large segment of the population to use the described caloric management system.

Further features and advantages of the invention will be apparent from the description below.

### BRIEF DESCRIPTION OF THE DRAWINGS

The invention is herein described, by way of example only, with reference to the accompanying drawings, wherein:

FIG. 1 is a block diagram illustrating one form of caloric management system constructed in accordance with the present invention;

FIG. 2 is a block diagram illustrating the main components of the personal user devices in the system of FIG. 1;

FIG. 3 is a block diagram illustrating the data processor at the remote location for serving a plurality of personal user devices;

FIG. 4 is a flowchart illustrating the basic operations involved in implementing a caloric management system in accordance with the present invention; and

FIG. 5 illustrates an example of the display screen on a personal user device for using the balance log for meal logging in accordance with the present invention.

### DESCRIPTION OF A PREFERRED EMBODIMENT

Overview of Construction and Operation

In the preferred embodiment of the invention described below, each time a user consumes a meal, the user enters into the system the food and beverage constituents of the meal by voice messages dictated into a personal user device communicating via a public network, such as the Internet or the telephone system, with a central website. The website includes a data processor or server which contains an application program with deep voice recognition software having the capability of converting the voice messages into digital signals representative of the food and beverage constituents of the meal, and entering their values into a data file for the user. The data processor at the Internet site may also interactively interrogate the user to better define the food constituents and the portion sizes.

The items consumed, and the portion sizes thereof, are dictated into the microphone of the personal user device, such as a PDA having Internet access capability, a cellular telephone, a Palm-like device having cellular capability, a laptop computer connected wirelessly to a local network which in turn connects to the Internet, a personal computer, or an interactive TV interface. An interactive TV interface could include an audio-to-digital converter as part of a remote control which accesses a set top box, or an interactive TV interface built into a TV receiver. The digitized voice message would then be transmitted to the data processor at the website via the Internet.

The powerful voice recognition capability that can be built into an application program supported on a server computer, serving as the data processor at the website, can easily analyze the audio messages and generate digital signals that can be entered into the database of the web.

In the described preferred embodiment of the invention, each user has a personal file on the website which can be accessed by the PDA, personal computer or interactive TV, to allow the user to determine the user's total nutritional intake or caloric consumption over a period of time, such as a day or week, and the user's caloric balance, by entering signals representing the user's basic metabolic rate and the user's activities, both of which can be converted into caloric expenditure. In an alternative embodiment of the invention, the user may carry an audio recorder to capture voice messages relative to food consumption or activity, and may download the stored information into the Internet by placing the recorder in a docking station, or transmitting the recorded messages through an interactive TV interface or the like.

The data processor at the website maintains a separate file for each user in which the data processor records the daily food consumption expressed in caloric values, and information relating to the user's resting metabolism and activities generating caloric expenditures, to maintain a caloric balance indicating the net caloric intake or expenditure by the user during a daily period. This is compared to goals for caloric expenditure in order to achieve weight goals, and messages may be transmitted to the user via the Internet relative to the user's success in meeting the weight goals. The messages may include recommendations for adjustments in diet or exercise in order to achieve the goals.

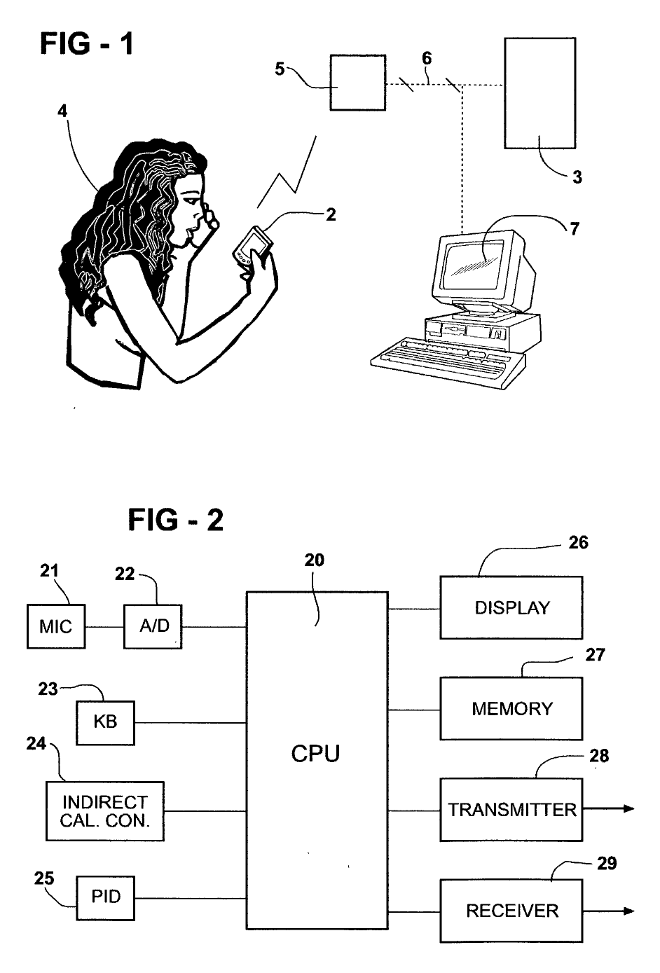

The Overall System Illustrated in FIG. 1

FIG. 1 is a schematic diagram illustrating the overall system. It includes a plurality of personal user devices, each generally designated 2, all communicating with a data processor 3 at a remote location for assisting the users of the system to implement a caloric management program personal to each user. As will be described more particularly below, each personal user device 2 is preferably a portable handheld unit, such as a handheld telephone or PDA. Each portable handheld unit includes a microphone and an analog/digital converter generating digital signals representative of the audio output, and a two-way wireless communication capability with the data processor 3 at the remote location. The data processor 3 is preferably a server at an Internet website.

Thus, as shown in FIG. 1, each user of the system, schematically indicated at 4, utilizes the personal user device 2 to establish a wireless connection through a local Internet cell 5 to the data processor 3 via the Internet 6. Data processor 3, preferably a website server as indicated above, may also be accessed through personal computers 7 and the like via an Internet connection 8.

The Personal User Device 2 Illustrated in FIG. 2

FIG. 2 schematically illustrates the construction of each of the personal user devices 2 in the system of FIG. 1. Thus, each personal user device 2 includes a CPU 20 having one input from a microphone 21 via an analog-to-digital converter 22 for inputting speech information, and a keyboard 23 for manually inputting other information.

The speech information may include food consumption in the form of various food items, and the quantities thereof, as consumed by the user. Such information is used for computing the caloric intake value for a particular (e.g., daily) time period.

Microphone 21 may also be used for inputting information relating to various physical activities performed by the user and the period of time of performance of such activities. The latter information is used for computing the activity-related energy expenditure (AEE) of the user over the particular time period.

In order to compute also the resting energy expenditure (REE) of the user over the particular time period, it is necessary to input the resting metabolism rate of the individual for the particular time period. This may be done by inputting, either via microphone 21 or keyboard 23, the sex, age and weight of the user, to enable a rough estimate of the resting metabolism rate to be computed according to the known Harris-Benedict Equation. However, for a more accurate determination of the resting metabolism rate, the personal user device 2 preferably also includes a connector 24 for connecting an indirect calorimeter thereto, such as described in the above-cited co-pending patent application Ser. No. 09/630,398, the contents of which are incorporated herein by reference.

The inputted resting energy expenditure (REE) derived from the resting metabolism rate, is added to the inputted activity-related energy expenditure (AEE) derived from the physical activities. The sum is used, together with the time periods thereof as inputted into the personal user device 2, for producing the total caloric expenditure (TEE) value for the particular time period.

The portable user device 2 further includes a personal identification device 25 which produces a personal identification signal. This signal is also inputted into the CPU 20 to identify the respective personal user device.

As also shown in FIG. 2, the personal user device 2 also includes a display screen 26 for displaying various data as inputted into the device, as processed by the device, and/or as received from the data processor 3 at the remote location. The inputted and processed data is stored in a memory 27.

A portable audio recorder (not shown) may be used for recording the identification and quantity of the food items as consumed by the user, and/or the physical activities as performed by the user. Such information may be recorded in the recorder in a real-time manner and downloaded via microphone 21 into memory 27 of the personal user device 2 at convenient time intervals.

The personal user device 2 further includes a transmitter 28 and a receiver 29 for enabling two-way communication with the data processor 3 at the remote location, preferably in a wireless manner and via the Internet as described earlier.

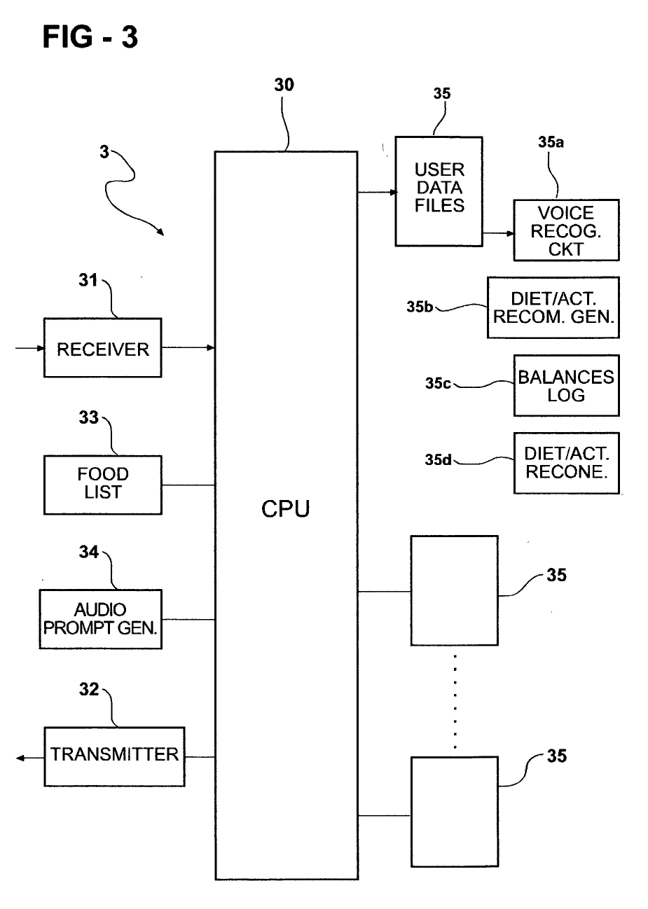

The Remotely-Located Data Processor Illustrated in FIG. 3

FIG. 3 illustrates the data processor 3 at the remote location for servicing the plurality of personal user devices 2. As indicated earlier, data processor 3 may be a website server connected to the Internet.

As shown in FIG. 3, data processor 3 includes a CPU 30 having a receiver 31 and a transmitter 32 for providing two-way communication via the Internet with the plurality of personal user devices 2.

Data processor 30 further includes a lookup table 33 containing various food items that may be consumed by the users, and the caloric value of a predetermined quantity of each such food item. Since such a lookup table is not user dependent, a single table may be used by the data processor for all the users, that is, for determining the equivalent caloric values of each quantified food item consumed by any of the users as inputted via their respective personal user devices 2.

Data processor 3 illustrated in FIG. 3 may optionally include an audio prompt generator 34. Such a generator, upon receiving a transmission from a personal user device 2, enables the data processor to transmit back to the respective personal user device an inquiry regarding the transmission by the latter device. Such an inquiry would require a response from the respective personal user device.

For example, if a personal user device 2 establishes communication with the data processor 3 without identifying itself, the audio prompt generator 34 may transmit back to the personal user device 2 a message asking it to identify itself. Also, after identification, the audio prompt generator may send a message to the personal user device 2 asking for better identification of the type of food consumed and/or the quantity thereof. Such inquiries may be done sequentially. For example, the audio prompt generator may message “please identify the first food eaten”; and if the response is “cheeseburger”, the audio prompt generator may then inquire “large or small?”, etc.

Data processor 3 at the remote location also includes a user data file, generally designated 35, for each personal user device 2. Each user data file 35 includes a number of components which are related to or dependent upon the specific user.

Thus, as shown in FIG. 3, each user data file 35 includes a voice recognition circuit 35a for analyzing the electrical speech signals received from the respective personal user device 2 to identify the quantitized food items, and the various physical activities, as voice-inputted by the user in the user's respective personal user device 2. While the equivalent caloric value of each quantitized food item consumed by the user can be taken from the common food lookup table 33, the equivalent caloric value of a physical activity that may be performed by a user is dependent on the specific user; therefore, a physical activity lookup table 35b is provided in each user's data file 35 personal to the respective user.

Each user data file 35 further includes a balance log 35c which maintains a running balance of the caloric intake value represented by the food items consumed by the user, less the caloric expenditure value representing the total energy expenditure of the user, for the particular time period. As indicated earlier, the total energy expenditure (TEE) includes the activity-related energy expenditure (AEE) derived from the activity lookup table 35b, and the resting energy expenditure (REE) derived from the resting metabolism rate of the user, as measured by an indirect calorimeter or as approximately computed by inputs of age, sex and weight of the user.

Each of the user data files 35 may also include a diet/activity recommendations generator 35d. Thus, as will be described more particularly below, each user may input into the user's respective file 35, in the data processor 3 at the remote location, a caloric management program desired to be followed by the user. As the user progresses with the caloric management program, the user inputs into the respective user data file foods consumed, physical activities performed, and other information enabling a determination of the user's resting metabolism rate. The data processor continuously compares the user's progress with respect to the desired caloric management program, and periodically, or whenever desired, generates recommendations as to the future diet and/or future physical activities of the user in order to better implement the inputted caloric management program by the user.

Example of Operation of the System

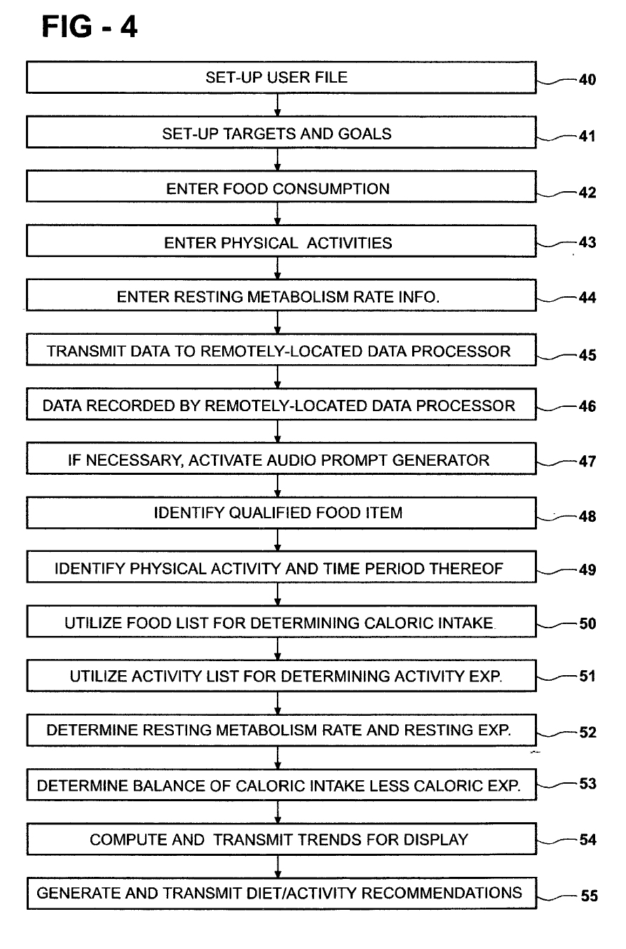

FIG. 4 is a flowchart illustrating one manner of using the system of FIGS. 1-3 for implementing a caloric management program personal to the respective user. The caloric management program may be designed, for example, for weight control purposes, e.g., to achieve a desired weight loss within a specified period of time.

According to block 40 in FIG. 4, the user, using a personal user device 2, first sets up a user data file in the data processor 3 at the remote location, e.g. a website server connected to the Internet. This may be done by using the personal user device 2 for communicating with the data processor 3, and identifying the particular user with the personal identification device 25 of the personal user device 2 as well as providing other identification particulars that may be desired. At that time, or at any time thereafter, the user may also set up targets and goals of a caloric management program desired to be followed by the user (block 41).

As the caloric management program proceeds, the user enters the food consumption by identifying the various food items, and the quantities, as consumed by the user (block 42). This information is conveniently voice-inputted via the microphone 21 of the personal user device 2, which microphone converts the speech sounds by the user to electrical speech signals. Such signals are converted to digital form by A/D converter 22 before being fed to the CPU 20 of the personal user device 2.

The user may also input, via microphone 21 and A/D converter 22, various physical activities performed by the user during the respective time period (block 43). In addition, the user may input information enabling a determination to be made of the resting metabolism rate of the user (block 43) by connecting the personal user device 2 to an indirect calorimeter 23; alternatively, an approximation of the resting metabolism rate may be determined according to the known Harris-Benedict Equation by inputting the sex, weight and age of the user via microphone 21 or keyboard 23, as briefly described above.

All the foregoing information may be inputted into the respective personal user device 2 in a real-time manner as each event occurs, and may immediately be transmitted (block 45) by that device to the data processor 3 at the remote location for processing. Alternatively, the foregoing information may be inputted into the personal user device 2 in a real-time manner, or at more convenient times, and temporarily stored in the personal user device for downloading to the remotely located data processor at convenient times.

Another alternative is to record the voice messages in an audio recorder (not shown), such as a small handheld dictating machine, and download those voice messages into the memory 27 via the microphone 21 at convenient time intervals.

After the foregoing information has been inputted into the respective personal user device 2, it may be transmitted by the two-way wireless communication system to data processor 3 at the remote location. For example, this may be done by including a “Send” button on the personal user device which is depressed to transmit the inputted information.

This transmitted information is received by the data processor 3 at the remote location (block 46) and is processed in the following manner.

First, if further information is needed by the data processor, the audio prompt generator 34 is actuated to obtain the further information (block 47). For example, if the transmitting personal user device 2 did not identify itself, the audio prompt generator transmits back to that device an inquiry requesting it to identify itself. If the spoken message cannot be readily identified by the voice recognition circuit within the data processor, the audio prompt generator may transmit back to the device a message requesting the voice message to be repeated, or to be differently worded. If the voice message did not specify the quantity of a food item consumed, or the time period of a physical exercise, this information may also be requested by the audio prompt generator.

Assuming that the data processor received all the necessary information for processing, it analyzes the electrical speech signals to identify the quantitized food item (block 48) and/or the physical activity and time thereof (block 49) represented by such electrical speech signals. Data processor 3 utilizes the common food lookup table 33 for determining the caloric intake (block 50), and the physical activity lookup table 35b for the respective user (block 51) for determining the activity-related expenditure energy (AEE) represented by such voice messages. The data processor 3 also determines the resting metabolism rate (block 52) from the input of the indirect calorimeter, or from the age, sex and weight input, and determines therefrom the resting expenditure energy (REE).

Data processor 3 then determines, for the respective user, the balance of caloric intake minus caloric expenditure (block 53). This information is transmitted back to the respective personal user device 2 for display thereon. Data processor 3 may also compute, and transmit back to the respective personal user device 2, trends comparing the actual progress with the caloric management program inputted by the user (block 54), for display on the user's personal user device 2, so as to reinforce the user's progress if the progress is satisfactory, and to alert the user if the progress is not satisfactory. Data processor 3 may also generate recommendations concerning diet and/or physical activity changes desirable in order to better enable the user to implement the desired caloric management program (block 55).

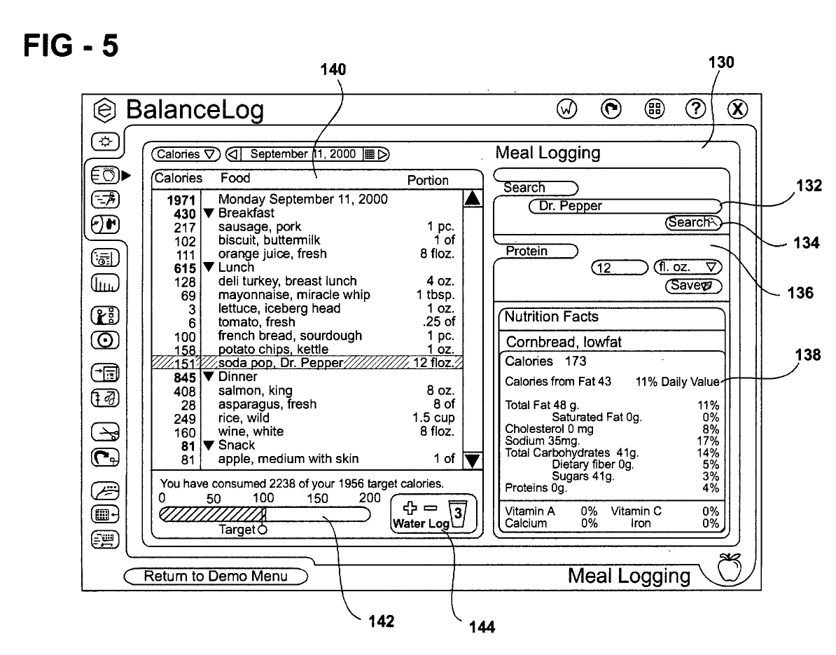

The Balance Log 35e Illustrated in FIG. 5

The balance log 35e in the user data file 35 for the respective personal user device 2 may be of the type disclosed in U.S. patent application Ser. No. 09/976,967, assigned to the assignee of the present application, the contents of which are incorporated herein by reference. FIG. 5 illustrates an example of such a balance log, which corresponds to FIG. 16 in that patent application.

When using the balance log for meal logging as illustrated in FIG. 5, the user enters the name of a food item into a search form 132 and selects the search icon 134. Food items matching the entered text are retrieved along with nutrition facts, and are displayed in window 138. The portion size can be adjusted and saved in association with the retrieved food using window 136. By searching a number of food items consumed during a meal, the user builds up a diet log of meals eaten, which is displayed in window 140. The method provides a visual indication of the progress towards calorie intake goals using a graphic display 142, and a log of water consumption using a graphic display 144.

The diet/activity recommendations generator 35d can use the information generated in the balance log file 35c to recommend activities or diet changes that can result in the user achieving predetermined weight, nutritional or exercise goals. These recommendations are made available on the balance log file and may be transmitted back to the user via the Internet.

When the personal user device 2 employed incorporates a display screen 26, the balance log file may be transmitted, typically in summary form, to the personal user device 2. Alternatively, the user may access the data processor 3 through a personal computer 7 (FIG. 1) to receive the complete balance log display. The balance log illustrated in FIG. 5 is a typical display

The user may alternatively establish communication with the data processor through a cellular telephone or an interactive TV system. The interactive TV system would be equipped with a voice-to-digital converter in order to transmit the digitized voice messages of the user to the server. The audio/digital converter could be in the remote control of the digital interactive TV system, or it could be incorporated in the set top box or built into the television receiver. In the latter case, the user would not use a remote unit but would simply speak to the interface unit in order to provide the voice signals to the server.

Examples of Variations

While the invention has been described with respect to a preferred embodiment, it will be appreciated that this is set forth merely for purposes of example, and that many variations may be made. For example, if a less powerful voice recognition capability is acceptable for any particular application (e.g., requiring recognition of a very limited vocabulary), the voice recognition circuit used may not be user-dependent, such that a common voice recognition circuit could be used for all the user data files. Similarly, in some cases a common activity lookup table may be acceptable for all users, or for groups of users divided into, various categories, such as sex, age, weight, etc., so that each user need not have a separate lookup table for physical activities. In addition, where minimum cost is not a primary requirement, some or all of the personal user devices could each be provided with its own voice recognition circuit voice-trained for the particular user and/or, with its own physical activity lookup table tailored for the particular user.

Many other variations, modifications and applications of the invention will be apparent.

1. A caloric management system for assisting a plurality of users to implement a caloric management program personal to each user, comprising:
(a) a plurality of personal user devices each including:
(i) input means including a microphone for converting speech sounds by the respective user to electrical signals for outputting from the respective personal user device;
(ii) a personal identification device for applying a personal identification signal in the output of the respective personal user device;
(iii) a display for displaying information; and
(iv) a two-way communication system for transmitting the output of the personal user device to a remote location serving said plurality of personal user devices, and for receiving information from said remote location;
(b) a voice recognition circuit for analyzing the outputs of the microphones of the plurality of personal user devices to identify quantified food items represented thereby;
(c) and a data processor at said remote location including:
(i) a two-way communication system for receiving said output from each personal user device;
(ii) a food lookup table containing data representing a number of quantified food items and the caloric intake value of each such food item; and
(iii) a user data file for each personal user device, each of said user data files including a computer for computing the total caloric intake value of the quantified food items identified in a particular time period from the respective personal user device and for transmitting information regarding said total caloric intake value to the respective personal user device for display thereon.

2. The system according to claim 1, wherein said voice recognition circuit is included in the data processor at said remote location.

3. The system according to claim 1, wherein said voice recognition circuit is user-dependent and is included in each of said user data files in the data processor at said remote location.

4. The system according to claim 1, wherein said voice recognition circuit is included in each of said personal user devices.

5. The system according to claim 1, wherein said input means in each of said personal user devices also enables the respective user to input a caloric management program into its respective user data file in the data processor at the remote location; and wherein the information transmitted by the computer in the respective user data file also includes information comparing the total caloric intake value for the respective time period with the caloric management program inputted into the respective user data file.

6. A caloric management system for assisting a plurality of users to implement a caloric management program personal to each user, comprising:
(a) a personal user device for each user including:
(i) input means including a microphone for receiving speech sounds by the user and for converting such speech sounds to electrical speech signals;
(ii) a personal identification device for producing a personal identification signal for the respective personal user device;
(iii) a display for displaying information; and
(iv) a two-way communication system for transmitting said electrical speech signals and said personal identification signal to a remote location serving a plurality of personal user devices, and for receiving information from said remote location;
(b) and a data processor at said remote location including:
(i) a two-way communication system for receiving said electrical speech signals and personal identification signal from each personal user device;
(ii) a food lookup table containing data representing a number of quantified food items and the caloric intake value of each such food item; and
(iii) a user data file for each personal user device; each of said user data files including:
(a) a voice recognition circuit for analyzing said electrical speech signals received from the respective personal user device to identify the quantified food item represented thereby; and
(b) a computer for computing the total caloric intake value of said quantified food items identified in a particular time period from the respective personal user device, and for transmitting information regarding said total caloric intake value to the respective personal user device for display thereon.

7. The system according to claim 5,
wherein at least some of said user data files in the data processor at the remote location also include an activity lookup table containing data representing a number of physical activities and the caloric expenditure value of each such physical activity when performed by the respective user per unit time;
wherein said voice recognition circuit in each user data file having an activity lookup table also analyzes said electrical speech signals received from the respective personal user device to identify physical activities represented thereby and the period of time thereof;
and wherein said computer in each user data file having an activity lookup table also computes the caloric expenditure value for the particular physical activity and time period, and includes the caloric expenditure value in the information transmitted to the respective personal user device.

8. The system according to claim 7,
wherein said input means of each personal user device also enables inputting resting metabolism rate information which is converted to electrical metabolism signals also transmitted to said data processor at the remote location;
and wherein said computer in each user data file having an activity lookup table also analyzes said electrical metabolism signals for computing the caloric expenditure value corresponding to the resting metabolism rate for the respective time period, and includes such information in computing the caloric expenditure value transmitted to the respective personal user device.

9. The system according to claim 8, wherein said input means includes a connector for an indirect calorimeter for inputting said resting metabolism rate information.

10. The system according to claim 8, wherein said input means enables inputting information regarding the sex, age and weight of the user; and said computer in the user data file for the respective personal user device computes an estimate of the resting metabolism rate from said inputted information.

11. The system according to claim 7,
wherein said input means in each of said personal user devices also enables inputting a caloric management program into its respective user data file in the data processor at the remote location;
and wherein said computer in the respective user data file at the remote location also generates diet and/or activity recommendations which are included in the information transmitted to the respective personal user device to better enable the user to implement the inputted caloric management program.

12. The system according to claim 6, wherein said data processor at the remote location also includes an audio prompt generator which, upon receiving a transmission from a personal user device, enables the data processor to transmit back to the respective personal user device an inquiry regarding the transmission by the personal user device, which inquiry requires a response from the respective personal user device.

13. The system according to claim 6, wherein at least some of said personal user devices are coupleable to portable hand-carried recording devices which include audio recorders for recording the identification of the quantified food items as consumed by the user and for downloading into said personal user devices at convenient time intervals.

14. The system according to claim 6, wherein said two-way communication system between the personal user devices and the data processor at the remote location is via the Internet.

15. A method of assisting a plurality of users to implement a caloric management program personal to each user by means of a caloric management system according to claim 1, said method comprising:
utilizing the personal user devices for inputting speech messages identifying various quantified food items consumed by the users and for producing outputs from said personal user devices identifying the quantified food items consumed by the respective users within a particular time period;
transmitting from each of said personal user devices to said data processor at the remote location the output, including the personal identification signal, of the respective personal user device;
computing in each user data file at the remote location the total caloric intake value of said quantified food items identified from the output of the respective personal user devices during a particular time period; and
transmitting information regarding said total caloric intake value to the respective personal user devices for display thereon.

16. The method according to claim 15,
wherein the users also input, via their respective personal user devices, a caloric management program into the respective user data file in the data processor at the remote location;
and wherein the computer in the respective user data file in the data processor at the remote location transmits information comparing the total caloric intake value for the respective time period with the caloric management program inputted into the respective user data file.

17. The method according to claim 15,
wherein the users also input, via their respective personal user devices, physical activities by the users and the time periods for each such physical activity;
wherein the user data file in the data processor at the remote location for the respective personal user device also includes an activity lookup table containing data representing a number of physical activities and the caloric expenditure value of each such physical activity when performed by the respective user per unit time;
wherein said voice recognition circuit also identifies physical activities inputted into the personal user device and the period of time thereof;
and wherein said computer in each of said user data files also computes the caloric expenditure value for the particular physical activity and time period, and provides the caloric expenditure value, in the information transmitted to the respective personal user device.

18. The method according to claim 17,
wherein the users also input, via their respective personal user devices, the resting metabolism rate of the respective user for the respective time period by inputting the age, sex and weight of the user;
and wherein the computer in each user data file computes the resting energy expenditure and utilizes such information in computing the caloric expenditure value transmitted to the respective personal user device.

19. The method according to claim 17,
wherein the users also input, via their respective personal user devices, the resting metabolism rate of the respective user for the respective time period by inputting into the respective personal user device the output of an indirect calorimeter;
and wherein the computer in each user data file computes the resting energy expenditure and utilizes such information in computing the caloric expenditure value transmitted to the respective personal user device.

20. The method according to claim 17,
wherein the users also input, via their respective personal user devices, a caloric management program into the respective user data file in the data processor at the remote location;
and wherein the computer in the respective user data file in the data processor at the remote location computer, and transmits to the respective personal user device, information comparing the total caloric intake value and caloric expenditure value for the respective time period with the caloric management program inputted into the respective user data file.

21. The method according to claim 17,
wherein the users also input, via their respective personal user devices, a caloric management program into the respective user data file at the remote location;
and wherein the computer in the respective user data file at the remote location also generates diet and/or activity recommendations which are included in the information transmitted to the respective personal user device to better enable the user to implement the inputted caloric management program.

22. The method according to claim 15, wherein said data processor at the remote location also includes an audio prompt generator which, upon receiving a transmission from a personal user device, enables the data processor to transmit back to the respective personal user device an inquiry regarding the transmission by the personal user device, which inquiry requires a response from the respective personal user device.

23. The method according to claim 15, wherein at least some of said personal user devices are coupleable to portable hand-carried recording devices which include audio recorders for recording the identification of the quantified food items as consumed by the user and for downloading into said personal user devices at convenient time intervals.

24. The method according to claim 15, wherein said two-way communication system between the personal user devices and the data processor at the remote location is via the Internet.
